(19) United States
(12) Patent Application Publication
(10) Pub. No.: US 2021/0100663 A1
(43) Pub. Date: Apr. 8, 2021

(54) MIXED VARIABLE DECODING FOR NEURAL PROSTHETICS

(71) Applicant: California Institute of Technology, Pasadena, CA (US)

(72) Inventors: Carey Y. Zhang, Pasadena, CA (US); Tyson Aflalo, Pasadena, CA (US); Richard A. Andersen, Pasadena, CA (US)

(73) Assignee: California Institute of Technology, Pasadena, CA (US)

(21) Appl. No.: 17/121,218

(22) Filed: Dec. 14, 2020

Related U.S. Application Data
(63) Continuation of application No. 15/850,625, filed on Dec. 21, 2017.
(60) Provisional application No. 62/437,879, filed on Dec. 22, 2016.

(51) Int. Cl. A61F 2/72; G06F 3/01; G06N 3/06; G06N 20/00; A61F 4/00

(52) U.S. Cl. CPC A61F 2/72; G06F 3/015; A61F 2002/704; G06N 20/00; A61F 4/00; G06N 3/061

(57) ABSTRACT

In an embodiment, the invention relates to neural prosthetic devices in which control signals are based on the cognitive activity of the prosthetic user. The control signals may be used to control an array of external devices, such as prosthetics, computer systems, and speech synthesizers. Data obtained from a 4×4 mm patch of the posterial parietal cortex illustrated that a single neural recording array could decoded movements of a large extent of the body. Cognitive activity is functionally segregated between body parts.

### CROSS REFERENCE TO RELATED APPLICATIONS

This application claims the benefit of priority under 35 U.S.C. § 120 as a continuation of U.S. application Ser. No. 15/850,625, filed Dec. 21, 2017 and also claims the benefit of priority from U.S. Provisional Application Ser. No. 62/437,879, filed Dec. 22, 2016, the entirety of which is hereby incorporated by reference.

### STATEMENT REGARDING FEDERALLY SPONSORED RESEARCH OR DEVELOPMENT

This invention was made with government support under Grant No. EY015545 awarded by National Institutes of Health. The government has certain rights in the invention.

### BACKGROUND

Many limb prostheses operate in response to muscle contractions performed by the user. Some prostheses are purely mechanical systems. For example, a type of lower limb prosthesis operates in response to the motion of the residual limb. When the user's thigh moves forward, inertia opens the knee joint of the prosthesis, an artificial shin swings forward, and, when the entire structure locks, the user may pass his or her weight over the artificial leg. Other prostheses may incorporate electric sensors to measure muscle activity and use the measured signals to operate the prosthesis.

Such prostheses may provide only crude control to users that have control over some remaining limb musculature, and hence may not be useful for patients with spinal damage. For these patients, it may be desirable to measure precursor signals coded for limb movement in the patient's brain, and then decode these signals to determine the intended movement and/or target. A similar approach can be applied to patients with paralysis from a range of causes including peripheral neuropathies, stroke, and multiple sclerosis. The decoded signals could be used to operate pointing external devices such as a computer, a vehicle, or a robotic prosthesis.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1A illustrates a timeline showing an example of a delayed movement paradigm experimental cues to subjects. Subjects were cued as to what kind of movement to perform (e.g. imagine/attempt left/right hand/shoulder) and then cued to perform the movement after a brief delay.

FIGS. 1B-1E depict plots of firing rates of example single units over time (mean±sem), separated by cognitive motor strategy (attempt, imagine, speak) and side (left or right) for hands.

FIG. 2A depicts a graph showing the fraction of units in the population tuned for each condition in the Delay and Go phases, separated by body part and body side, shown as the bootstrapped 95% confidence intervals. A unit was considered tuned to a condition if the beta value of the linear fit for the condition (from the linear analysis described in the methods section) was statistically significant (p<0.05).

FIG. 2B depicts graphs showing the magnitudes of the units' tuning to each condition in the Delay and Go phases, as defined by the area under the receiver operating characteristic curve (AUC) between Delay/Go and ITI activity, separated by body parts. Intervals represent the 95% confidence intervals of the magnitudes trial by trial. Only units with significant AUC, as determined by a Wilcoxon rank sum test (p<0.05), were included. (Att R=Attempt Right, Att L=Attempt Left, Imag R=Imagine Right, Imag L=Imagine Left, Spk R=Speak Right, Spk L=Speak Left).

FIGS. 3A-3D depict possible organizational models of neural representations. FIG. 3(A) depicts a diagram illustrating an organizational model where each of the eight movement conditions have anatomically separate representations, i.e distinct, non-overlapping networks. (ALH=Attempt Left Hand, ILH=Imagine Left Hand, ARH=Attempt Right Hand, IRH=Imagine Right Hand, ALS=Attempt Left Shoulder, ILS=Imagine Left Shoulder, ARS=Attempt Right Shoulder, IRS=Imagine Right Shoulder). FIG. 3B depicts a diagram illustrating a model where some networks are subordinate to other, e.g. imagined movements being subsets of attempted movements. FIG. 3C depicts a diagram illustrating a model where all the variables (body part, body side, and strategy) are indiscriminately mixed within the neural population. Neurons in this model would be expected to exhibit mixed selectivity, showing tuning to various conjunctions of variables. FIG. 3D depicts a diagram illustrating a model where hand and shoulder movement representations are functionally segregated, despite sharing the same neural population, and the other variables (body side and strategy) are mixed within each functional representation. Neurons in this model would still show mixed selectivity to the various variables but in such a way that the representation of body side and strategy would not generalize from one body part to another. This model is consistent with the results observed in this study. Note that solid lines in this diagram indicate anatomical boundaries of neural populations while dotted lines indicate functional boundaries/segregation.

FIGS. 4A-4H illustrate graphs showing that some units are strongly tuned to even the relatively less well represented variables. FIGS. 4A-B depict distribution of the degree of specificity to the imagine or attempt strategies in the population during trials using different sides, showing only units responsive to one or both strategies. FIGS. 4C-D depict distribution of the degree of specificity to the left or right side in the population for different strategies. FIGS. 4E-F depict distribution of the degree of specificity to the hand or shoulder in the population during trials using different sides. FIGS. 4G-H depict distribution of the degree of specificity to attempted/imagined movements compared to speaking.

FIGS. 5A-5B depict graphs showing how units tuned to one condition are more likely tuned to conditions with more shared traits. FIG. 5A depicts the similarity in neural populations between movements differing by one, two, and all three traits (strategy, side, body part) separated into the delay and movement phases. Similarity measured as the average correlation in the normalized firing rates between pairs of movement conditions. Higher correlations in yellow and lower correlations in blue. (ALH=Attempt Left Hand, ILH=Imagine Left Hand, ARH=Attempt Right Hand, IRH=Imagine Right Hand, ALS=Attempt Left Shoulder, ILS=Imagine Left Shoulder, ARS=Attempt Right Shoulder, IRS=Imagine Right Shoulder). FIG. 5B depicts the correlations between four movement types: left and right movements (averaged across both strategies), and speaking controls left and right. (SL=Speak Left, SR=Speak Right, ML=Movement Left, MR=Movement Right).

FIGS. 6A-6D depict bar graphs showing the least overlap for movements with different body parts (both above and below injury). FIG. 6A depicts the average correlation between movement conditions differing by exactly one task variable and grouped by the differing condition (e.g. for strategy, the average correlation of all movement condition pairs differing only by strategy). Intervals represent the 95% confidence intervals). FIG. 6B depicts that for movements above and below the level of injury, average correlation between movement conditions in the Delay and Go phases grouped by the number of differing traits (average of each cube in the movement phase). Intervals represent the 95% confidence intervals in the correlations. FIGS. 6C-D depict the same information as FIGS. 6A-B but with shoulder shrug movements replaced with shoulder abduction movements (a movement below the level of injury).

FIGS. 7A-H depict bar graphs showing that the representations of variables generalize across side and strategy but not body part. FIG. 7A depicts an example of how a decoder trained on Condition 1 data to classify between two variables would perform when tested on Condition 1 data (in-sample) and Condition 2 data (out-of-sample) if the representations of Condition 1 and Condition 2 were functionally segregated. The decoder would be expected to only perform well on Condition 1 (in-sample), and fail to perform above chance in Condition 2 (out-of-sample), not generalizing well. FIG. 7B is similar to 7A but in the case that the representations of Condition 1 and Condition 2 were functionally overlapping. The decoder would be expected to perform significantly above chance on both sets of data, generalizing well. FIG. 7C depicts the performance of decoders trained on data split by body part for classifying the body side. Blue bars represent the performance of the decoder trained on shoulder movement data while orange bars represent the performance of the decoder trained on hand movement data. Horizontal axis labels represent which body part's data each decoder was tested on. Performance was measured as the fraction of trials accurately classified by the decoder, with in-sample performance determined by cross-validation. Asterisks represent performance significantly different from chance, as determined by a rank shuffle test. The red line represents chance performance level (0.5) while the green line represents perfect performance (1.0). FIG. 7D is similar to FIG. 7C, but with decoding strategy instead of body side. FIGS. 7E-F depict similar to FIG. 7C but with data split by body side and decoding for body part and strategy, respectively. FIGS. 7G-H depict similar to FIG. 7C but with data split by strategy and decoding for body side and body part, respectively.

FIG. 8. depicts a confusion matrix showing all movement variables decodable from the population. Confusion matrix showing the percent of the time a decoder trained to classify between the eight movement conditions misclassifies one condition as another. ALH=Attempt Left Hand, ILH=Imagine Left Hand, ARH=Attempt Right Hand, IRH=Imagine Right Hand, ALS=Attempt Left Shoulder, ILS=Imagine Left Shoulder, ARS=Attempt Right Shoulder, IRS=Imagine Right Shoulder, SL=Speak Left, SR=Speak Right, ML=Movement Left, MR=Movement Right).

FIG. 9 is an example of a block diagram of a neural prosthetic system utilizing cognitive control signals according to an embodiment of the present invention.

FIG. 10 is an example of a flowchart describing a technique for decoding and controlling a prosthetic utilizing cognitive control signals according to an embodiment of the present invention.

### DETAILED DESCRIPTION

All references cited herein are incorporated by reference in their entirety as if fully set forth. Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described.

In some embodiments, properties such as dimensions, shapes, relative positions, and so forth, used to describe and claim certain embodiments of the invention are to be understood as being modified by the term “about.”

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

### Cognitive Signals for Prosthetic Control

Previous studies that record the spike activity of neurons have focused primarily on deriving hand trajectory signals primarily, but not exclusively, from the motor cortex. Recordings from the cells are “decoded” to control the trajectories of a robotic limb or a cursor on a computer screen. Electroencephalogram (EEG) based signals have also been used to derive neuroprosthetic commands.

In an embodiment, cognitive control signals are derived from higher cortical areas related to sensory-motor integration in the parietal and frontal lobes. The primary distinction between cognitive signals from other types of signals, e.g., from the motor cortex, is not the location from which recordings are made, but rather the type of information being decoded and the strategy for using these signals to assist patients.

Cognitive signals are characterized as lying in the spectrum between pure sensory signals at the input, e.g., reflex due to light shined in an individual's eye, and motor signals at the output, e.g., signals used to execute a reach. Cognitive signals can result in neural activity in the brain even in the absence of sensory input or motor output. Examples of cognitive signals include abstract thoughts, desires, goals, trajectories, attention, planning, perception, emotions, decisions, speech, and executive control.

Experiments have recently been performed in monkeys in which reach intentions are decoded from neural activity in real time, and used to position a cursor on a computer screen—the so-called brain-control task. Arrays of electrodes were placed in the medial intraparietal area (MIP), a portion of the parietal reach region (PRR), area 5 (also in the posterior parietal cortex), and the dorsal premotor cortex (PMd).

PRR in non-human primates lies within a broader area of cortex, the posterior parietal cortex (PPC). The PPC is located functionally at a transition between sensory and motor areas and is involved in transforming sensory inputs into plans for action, so-called sensory-motor integration. The PPC contains many anatomically and functionally defined subdivisions.

Of particular interest in recent years are areas within the intraparietal sulcus that are involved in planning eye movements (the lateral intraparietal area, LIP), reach movements (PRR), and grasping (the anterior intraparietal area, AIP). PRR has many features of a movement area, being active primarily when a subject is preparing and executing a movement. However, the region receives direct visual projections and vision is perhaps its primary sensory input. Moreover, this area codes the targets for a reach in visual coordinates relative to the current direction of gaze (also called retinal or eye-centered coordinates). Similar visual coding of reaches has been reported in a region of the superior colliculus.

The use of cognitive signals also has the advantage that many of these signals are highly plastic, can be learned quickly, are context dependent, and are often under conscious control. Consistent with the extensive cortical plasticity of cognitive signals, the animals learned to improve their performance with time using PRR activity. This plasticity is important for subjects to learn to operate a neural prosthetic. The time course of the plasticity in PRR is in the range of one or two months, similar to that seen in motor areas for trajectory decoding tasks. Moreover, long-term, and particularly short-term, plasticity is a cognitive control signal that can adjust brain activity and dynamics to allow more efficient operation of a neural prosthetic device.

In addition, short term improvements in performance were achieved by manipulating the expected value of reward. The expected value of the probability of reward, the size of the reward and the type of reward were decoded from the activity in the brain control experiments. The finding of these signals in PRR is new, and parallels similar finding of expected value in nearby area LIP as well as other cortical and subcortical areas. This activity does not appear to be linked to attention since PRR is active selectively for reach plans and did not show an enhancement of activity to aversive reward trials.

The correlation of increased activity with increased expected reward is substantiated by behavioral data that showed a decrease in reaction times for the preferred rewards. Expected value is a necessary component of the neural system that mediates decision making. On the other hand, it is also possible that we are seeing motivational effects that are a direct consequence of expected value.

The decoding of intended goals is an example of the use of cognitive signals for prosthetics. Once these goals are decoded, then smart external devices can perform the lower level computations necessary to obtain the goals. For instance, a smart robot can take the desired action and can then compute the trajectory. This cognitive approach is very versatile because the same cognitive/abstract commands can be used to operate a number of devices. The decoding of expected value also has a number of practical applications, particularly for patients that are locked in and cannot speak or move. These signals can directly indicate, on-line and in parallel with their goals, the preferences of the subject and their motivational level and mood. Thus they could be used to assess the general demeanor of the patient without constant querying of the individual (much like one assesses the body-language of another). These signals could also be rapidly manipulated to expedite the learning that patients must undergo in order to use an external device. Moreover, different kinds of cognitive signals can be decoded from patients. For instance, recording thoughts from speech areas could alleviate the use of more cumbersome letter-boards and time consuming spelling programs. Or recordings from emotion centers could provide an on-line indication of the subjects' emotional state. Recording from areas of the brain involved in executive control, particularly cortical areas of the frontal lobe, can provide abstract associations of objects with actions as well as allow long-term planning signals to be utilized for control and programming.

The cognitive-based prosthetic concept is not restricted for use to a particular brain area. However, some areas will no doubt be better than others depending on the cognitive control signals that are required. Future applications of cognitive based prosthetics will likely record from multiple cortical areas in order to derive a number of variables. Other parts of the brain besides cortex also contain cognitive related activity and can be used as a source of signals for cognitive control of prosthetics. Finally, the cognitive-based method can easily be combined with motor-based approaches in a single prosthetic system, reaping the benefits of both. Likewise, the partially mixed-selectivity prosthetic concept is not restricted for use to a particular brain area. Future applications of partially mixed-selectivity based prosthetics will likely record from multiple cortical areas in order to derive a number of variables with different structures. These brain areas may include but are not limited to prefrontal cortex, premotor cortices, inferotemporal cortex, language cortices (Broca's and Wernicke's) amongst others.

An advantage of cognitive control signals is that they do not require the subject to make movements to build a database for predicting the subjects thoughts. This would of course be impossible for paralyzed patients. This point was directly addressed in off-line analysis by comparing the performance between “adaptive” and “frozen” databases. With the adaptive database, each time a successful brain-control trial was performed it was added to the database, and because the database was kept at the same number of trials for each direction, the earliest of the trials is dropped. Eventually only brain-control trials are contained within the database. In the case of the frozen database, the reach data was used throughout the brain-control segment. Both decodes were performed with the same data and both databases produce the same performance. Thus paralyzed patients can be simply asked to plan to make a reach and this planning activity can be used to build a database even though the patients cannot actually reach.

Signals related to reward prediction are found in several brain areas. PRR cells are more active and better tuned when the animal expects higher probability of reward at the end of a successful trial. PRR cell activity also shows a reward preference, being more active before the expected delivery of a preferred citrus juice reward than a neutral water reward. The expected value in brain-control experiments could be read out simultaneously with the goal using off-line analysis of the brain control trials. These experiments show that multiple cognitive variables can be decoded at the same time.

The partially mixed-selectivity prosthetic concept is not defined by a specific set of sensory, motor, or cognitive variables but, instead, is defined by the structured relationship between these variables as encoded in the neural population. Thus, the partially mixed-selectivity prosthetic concept should not be limited to any specific variables but should encompass any approaches that leverage the structure of how the neural code for variables are encoded with respect to each other as part of the decoding process. Variables might include, but are not limited to, behavioral goals, expected utility, error signals, motor control signals, spatial goal information, object shape information, object identity, spatial and feature attention, category membership, effort, body-state including posture, tactile, and peripersonal space monitoring, etc.

As described in U.S. Pat. No. 6,615,076, it has been found that the local field potentials (LFP) recorded in the posterior parietal cortex of monkeys contains a good deal of information regarding the animals' intentions. In an embodiment, the LFP may be recorded in addition to, or instead of, single unit activity (SU) and used to build the database(s) for cognitive signals and decode the subject's intentions. These LFP signals can also be used to decode other cognitive signals such as the state of the subject. Moreover, the same cognitive signals that can be extracted with spikes can also be extracted with LFPs and include abstract thoughts, desires, goals, trajectories, attention, planning, perception, emotions, decisions, speech, and executive control.

In one embodiment, an electrode may be implanted into the cortex of a subject and used to measure the signals produced by the firing of a single unit (SU), i.e., a neuron, in the vicinity of an electrode. The SU signal may contain a high frequency component. This component may contain spikes-distinct events that exceed a threshold value for a certain amount of time, e.g., a millisecond. Spikes may be extracted from the signal and sorted using known spike sorting methods.

Attempts have been made to use the spike trains measured from particular neurons to predict a subject's intended movements. The predicted intention could then be used to control a prosthetic device. However measuring a spike train with a chronic implant and decoding an intended movement in real time may be complicated by several factors.

In general, measuring SU activity with a chronic implant may be difficult because the SU signal may be difficult to isolate. An electrode may be in the vicinity of more than one neuron, and measuring the activity of a target neuron may be affected by the activity of an adjacent neuron(s). The implant may shift position in the patient's cortex after implantation, thereby changing the proximity of an electrode to recorded neurons over time. Also, the sensitivity of a chronically implanted electrode to SU activity may degrade over time.

LFP is an extracellular measurement that represents the aggregate activity of a population of neurons. The LFP measured at an implanted electrode during the preparation and execution of a task has been found to have a temporal structure that is approximately localized in time and space. Information provided by the temporal structure of the LFP of neural activity appears to correlate to that provided by SU activity, and hence may be used to predict a subject's intentions. Unlike SU activity, measuring LFP activity does not require isolating the activity of a single unit.

### Functional Segregation of Posterior Parietal Cortex

As discussed above, the posterior parietal cortex (PPC) of humans has historically been viewed as an association area that receives diverse inputs from sensory cortex, “associates” these inputs for processing more cognitive functions such as spatial awareness, attention and action planning, and delivers the outcomes of the associative process to more motor regions of the frontal cortex (Balint 1909, Holmes 1918, Mountcastle 1975, Ungerleider and Mishkin 1982). However, subsequent single neuron recording experiments with behaving non-human primates (NHPs) point to a systematic organization of functions in PPC (Andersen and Buneo 2002). Of particular interest to the current investigation, separate cortical areas around the intraparietal sulcus (IPS) have concentrations of neurons selective for saccades (lateral intraparietal area, LIP) (Andersen, Essick et al. 1987), reach (parietal reach region, PRR) (Snyder, Batista et al. 1997) and grasping (anterior intraparietal area, AIP) (Murata 2000). These data suggest that this part of the PPC, rather than being one large association region, is rather composed of a number of anatomically separated cortical fields that are specialized for intended movements that are effector-specific (eye, arm, hand).

More recent functional magnetic resonance imaging (fMRI) studies in humans have presented a mixed picture with some studies finding similar segregation for the types of intended movement in areas around the IPS (Astafiev 2003, Connolly, Andersen et al. 2003, Culham 2003, Prado 2005, Gallivan 2011, Gallivan, McLean et al. 2011) and other studies finding largely an intermixing of effectors (Levy 2007, Beurze 2009, Hinkley 2009, Heed, Beurze et al. 2011) as well as bimanually (Gallivan 2013). These findings provide evidence for a degree of distributed and overlapping representation of effectors on both sides of the body within PPC.

With the first chronic single neuron recordings of PPC in humans, similarities were found with the NHP studies. Neurons in human AIP are highly selective for different imagined grasp shapes while neurons in nearby Brodmann area (BA) 5 are not (Klaes 2015). However, the human neural recordings also pointed to some degree of distributed representation, with AIP neurons also selective for reach direction and with AIP and BA5 neurons being selective for reaches with either the left or the right limb or both (Aflalo, Kellis et al. 2015). While the inventors have discovered evidence that multiple effectors are encoded in the same anatomical region of cortex, these studies were carried out in separate sessions and thus the functional organization of multiple effectors within the same population of neurons remains unclear.

Pertinent to how different effectors are coded within PPC are recent results that address encoding strategies and their computational advantages in association cortices more generally. Neurons in prefrontal cortex and PPC (Rigotti, Barak et al. 2013, Raposo, Kaufman et al. 2014) exhibit what has been termed mixed selectivity (Fusi, Miller et al. 2016), a neural encoding scheme in which different task variables and behavioral choices are combined indiscriminately in a non-linear fashion within the same population of neurons. This scheme generates a high-dimensional non-linear representational code that allows for a simple linear readout of multiple variables from the same network of neurons (Fusi, Miller et al. 2016).

### Overview

The inventors examined the anatomical and functional organization of different types of motor variables within a 4×4 mm patch of human AIP. They varied movements along three dimensions: the body part used to perform the movement (hand versus shoulder), the body side (ipsilateral versus contralateral), and the cognitive strategy (attempted versus imagined movements). Each of these variables has been shown to modulate PPC activity (Gerardin, Sirigu et al. 2000, Andersen and Cui 2009, Heed, Beurze et al. 2011, Gallivan 2013). Thus they were able to look at how different categories of motor variables are encoded, and whether different variable types are treated in an equivalent manner (e.g. all variables exhibiting mixed-selectivity) or whether different functional organizations are found for different types of variables. Finally, the inventors compared the hand and shoulder movements to speech movements, a very different type of motor behavior.

Movements of the hand and shoulder are well represented in human AIP, whether they are imagined or attempted, or performed with the right or left hand. Single units were heterogeneous and coded for diverse conjunctions of different variables: there was no evidence for specialized subpopulations of cells that selectively coded one movement type. However, there was a significant overarching functional organization between the different motor variables. Body side and cognitive strategy were fundamentally different from body part at the level of neural coding. There was a high-degree of correlation between movement representations of the right and left side, within, but not between body parts. The same was true for cognitive strategy. Thus, body part acted as a superordinate variable that determined the structure of how the other subordinate variables were encoded. In contrast, the different body parts were better characterized as a mixed representation, with little obvious structure in how one body part was encoded in the population in relation to the other. Mixed-coding of some variables, but not others, argues in favor of PPC having a partially-mixed encoding strategy for motor variables. Finally, while AIP lacks anatomical segregation of body parts, mixed-coding between body parts leads to what we call functional segregation of body parts. Such segregation is hypothesized to enable multiple body parts to be coded in the same population with minimal interference. In some examples, once a superordinate variable of a population is determined or detected based on neural activity (e.g. body part), then the relevant subordinate variable (e.g. body side or cognitive strategy) can be determined based on the spatial location and intensity of the neural activity within that population.

### Systems and Methods

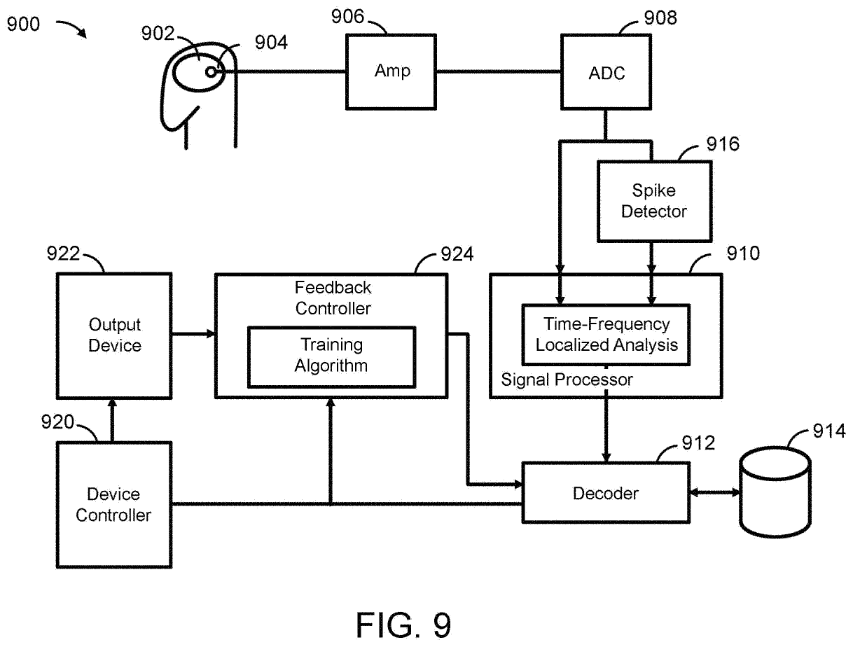

FIG. 9 illustrates a system 900 that uses cognitive signals to predict a subject's intended movement plan or other cognitive signal. The activity of neurons in the subject's brain 902 may be recorded with an implant 904. The implant 904 may include an array of electrodes that measure the action potential (SU) and/or extracellular potential (LFP) of cells in their vicinity. In one embodiment, micro-electro-mechanical (MEMS) technology may be used to prepare a movable electrode array implant. In alternate embodiments, the neural activity may be measured in forms other than electrical activity. These include, for example, optical or chemical changes, or changes in blood flow that may be measured by suitable measuring devices.

Neural activity measured with the implant 904 may be amplified in one or more amplifier stages 906 and digitized by an analog-to-digital converter (ADC) 908. In an embodiment, multiple implants may be used. Recordings may be made from multiple sites in a brain area, with each brain site carrying different information, e.g., reach goals, intended value, speech, abstract thought, executive control. The signals recorded from different implants may be conveyed on multiple channels.

The partially mixed-selectivity prosthetic concept is not restricted for use to a particular brain area. Future applications of partially mixed-selectivity based prosthetics will likely record from multiple cortical areas in order to derive a number of variables with different structures. These brain areas may include but are not limited to prefrontal cortex, premotor cortices, inferotemporal cortex, language cortices (Broca's and Wernicke's) amongst others.

The measured waveform(s), which may include frequencies in a range having a lower threshold of about 1 Hz and an upper threshold of from 5 kHz to 20 kHz may be filtered as an analog or digital signal into different frequency ranges. For example, the waveform may be filtered into a low frequency range of say 1-20 Hz, a mid frequency range of say 15-200 Hz, which includes the beta (15-25 Hz) and gamma (25-90 Hz) frequency bands, and a high frequency range of about 200 Hz to 1 kHz, which may include unsorted spike activity. In an alternate embodiment, the digitized signal may also be input to a spike detector 1316 which may detect and sort spikes using known spike sorting operations.

The digitized LFP signal, and the sorted spike signal if applicable, may be input to a signal processor 910 for time-frequency localized analysis.

The signal processor 910 may estimate the spectral structure of the digitized LFP and spike signals using multitaper methods. Multitaper methods for spectral analysis provide minimum bias and variance estimates of spectral quantities, such as power spectrum, which is important when the time interval under consideration is short. With multitaper methods, several uncorrelated estimates of the spectrum (or cross-spectrum) may be obtained from the same section of data by multiplying the data by each member of a set of orthogonal tapers. A variety of tapers may be used. Such tapers include, for example, parzen, Hamming, Hanning, Cosine, etc. An implementation of a multitaper method is described in U.S. Pat. No. 6,615,076, which is incorporated by reference herein in its entirety.

In an alternate embodiment the temporal structure of the LFP and SU spectral structures may be characterized using other spectral analysis methods. For example, filters may be combined into a filter bank to capture temporal structures localized in different frequencies. As an alternative to the Fourier transform, a wavelet transform may be used to convert the date from the time domain into the wavelet domain. Different wavelets, corresponding to different tapers, may be used for the spectral estimation. As an alternative to calculating the spectrum on a moving time window, nonstationary time-frequency methods may be used to estimate the energy of the signal for different frequencies at different times in one operation. Also, nonlinear techniques such as artificial neural networks (ANN) techniques may be used to learn a solution for the spectral estimation.

The processor 910 may generate a feature vector train, for example, a time series of spectra of LFP, from the input signals. The feature vector train may be input to a decoder 912 and operated on to decode the subject's cognitive signal, and from this information generate a high level control signal. The decoder 912 may use different predictive models to determine the cognitive signal. These may include, for example: probabilistic; Bayesian decode methods (such those described in Zhang, K., Ginzburg, I., McNaughton, B. L., and Sejnowski, T. J. (1998), Interpreting Neuronal population Activity by Reconstruction: Unified Framework with Application to Hippocampal place cells. J Neurophysiol 79:1017-1044); population vector models (such as those described in Lukashin, A. V., Amirikian, B. R., and Georgopoulos, A. P. (1996). A Simulated Actuator Driven by Motor Cortical Signals. Neuroreport 7(15-17):2597-2601); artificial neural networks, and linear discriminate classifiers.

Examples of how the partially-mixed selectivity may enhance prosthetic applications includes, but are not limited to:

Methods for the regularization of, or establishing priors on, decoder parameter values based on the discovered partially-mixed structure. Methods extend across particular mathematical realizations (Lu, Hirasawa et al. 2000, Maruyama and Shikita 2014, Razavian, Azizpour et al. 2014, e.g. Oktay, Ferrante et al. 2017). For example, decoder parameters learned for variables A and B are regularized to comport with the known mixing structure between A and B. This is to include methods for seeding initial decoder parameters across calibrated and uncalibrated variables based on discovered partially mixed structure between variables. For example, initializing parameters for variable A based on known parameters for variable B as determined by partially mixed structure.

Methods for updating decoding parameters for any subset of decodable variables X based on observed changes in the relationship between neural activity and decodable variables Y in order to preserve structure of partially mixed representations. For example, decoding parameter changes necessary for the loss of a neural channel discovered for variable A can be propagated for parameters B,C,D, etc. based on known relational structure between variables.

Methods to pattern external write-in signals to produce desired effect (e.g. sensory percept, motor action, feedback from learning/training etc.) that leverage partially-mixed structure of the recorded population. Write-in signals can include, but are not limited to, electrical microstimulation, optogenetic stimulation, and ultrasound. For example, stimulating to cause a hand sensation without causing a shoulder sensation. Sending direct neural feedback about one effector without affecting another effector's representations. Stimulating to cause a hand movement without causing a foot movement.

Decoding methods where the training or prediction stages utilize the known internal structure of how variables are encoded with respect to each other. This may include hierarchical classifications such as Bayesian hierarchical modeling or deep networks (i.e. deep Boltzman machines) (Silla and Freitas 2011, Salakhutdinov, Tenenbaum et al. 2013) in addition to other structured procedures (Bansal, Blum et al. 2004, Zhou, Chen et al, 2011, Li, Liu et al. 2014)

The decoder 912 may use a derived transformation rule to map a measured neural signal, s, into an action, a, for example, a target. Statistical decision theory may be used to derive the transformation rule. Factors in the derivations may include the set of possible neural signals, S, and the set of possible actions, A. The neuro-motor transform, d, is a mapping for S to A. Other factors in the derivation may include the intended target .theta. and a loss function which represents the risk associated with taking an action, a, when the true intention was θ. These variables may be stored in a memory device, e.g., a database 914.

In some examples two approaches may be used to derive the transformation rule: a probabilistic approach, involving the intermediate step of evaluating a probabilistic relation between s and θ and subsequent minimization of an expected loss to obtain a neuro-motor transformation (i.e., in those embodiments of the invention that relate to intended movement rather than, e.g., emotion); and a direct approach, involving direct construction of a neuro-motor transformation and minimizing the empirical loss evaluated over the training set. In terms of so-called “neural network” functions, the second approach may be regarded as defining a neural network with the neural signals as input and the target actions as output, the weights being adjusted based on training data. In both cases, a critical role is played by the loss function, which is in some sense arbitrary and reflects prior knowledge and biases of the investigator.

As described above, the measured waveform(s) may be filtered into a low frequency range of say 1-20 Hz, a mid frequency range of say 15-200 Hz, which includes the beta (15-25 Hz) and gamma (25-90 Hz) frequency bands, and a high frequency range of about 200 Hz to 1 kHz, which may include unsorted spike activity. The decoder 912 may decode a cognitive signal using the information in the gamma frequency band (25-90 Hz) of the LFP spectra and the SU spectra. The decoder 912 may decode logical signals using information in the gamma (25-90 Hz) and beta (15-25 Hz) frequency bands of the LFP spectra and the SU spectra. The logical information may include a decision to execute an action, e.g., a “GO” signal. The logical information may indicate that the subject is entering other states, such as cuing a location, preparing to execute an action, and scrubbing a planned action.

Once the decoder 912 maps the feature vectors from the signal processor 910 to an action, the decoder 912 may generate a high level signal indicative of the cognitive signal and transmit this signal to the device controller 920. The device controller 920 may use the signal to control the output device 922 to, e.g., mimic the subject's intended movement or perform another task associated with the cognitive signal. The output device may be, for example, a robotic limb, an animated limb or a pointing device on a display screen, or a functional electrical stimulation device implanted into the subject's muscles for direct stimulation and control.

The decoder 912 may need to be recalibrated over time. This may be due to inaccuracies in the initial calibration, degradation of the implant to spike activity over time, and/or movement of the implant, among other reasons.

In an embodiment, the decoder 912 may use a feedback controller 924 to monitor the response of the output device, compare it to, e.g., a predicted intended movement, and recalibrate the decoder 912 accordingly. The feedback controller 924 may include a training program to update a loss function variable used by the decoder 912.

Some error may be corrected as the subject learns to compensate for the system response based on feedback provided by watching the response of the output device. The degree of correction due to this feedback response, and hence the amount of recalibration that must be shouldered by the system 900, may depend in part on the degree of plasticity in the region of the brain where the implant 904 is positioned

The subject may be required to perform multiple trials to build a database for the desired cognitive signals. As the subject performs a trial, e.g., a reach task or brain control task, the neural data may be added to a database. The memory data may be decoded, e.g., using a Bayesian algorithm on a family of Haar wavelet coefficients in connection with the data stored in the database, and used to control the prosthetic to perform a task corresponding to the cognitive signal. Other predictive models may alternatively be used to predict the intended movement or other cognitive instruction encoded by the neural signals.

Indeed, there are a wide range of tasks that can be controlled by a prosthetic that receives instruction based on the cognitive signals harnessed in various embodiments of the present invention. Reaches with a prosthetic limb could be readily accomplished. A cursor may be moved on a screen to control a computer device. In another embodiment, the implant may be placed in the speech cortex, such that as the subject thinks of words, the system can identify that activity in the speech center and use it in connection with a speech synthesizer. In this embodiment, a database may first be built up by having a subject think of particular words and by detecting the accompanying neural signals. Thereafter, signals may be read in the speech cortex and translated into speech through a synthesizer by system recognition and analysis with the database. Alternatively, the mental/emotional state of a subject (e.g., for paralyzed patients) may be assessed, as can intended value (e.g., thinking about a pencil to cause a computer program (e.g., Visio) to switch to a pencil tool, etc.). Other external devices that may be instructed with such signals, in accordance with alternate embodiments of the present invention, include, without limitation, a wheelchair or vehicle; a controller, such as a touch pad, keyboard, or combinations of the same; and a robotic hand. As is further described in the ensuing Experimental Results, the system can also decode additional abstract concepts such as expected value. Still further applications for the system of the present invention can be readily identified and implemented by those of skill in the art.

Various implementations of the systems and techniques described here can be realized in digital electronic circuitry, integrated circuitry, specially designed ASICs (application specific integrated circuits), computer hardware, firmware, software, and/or combinations thereof. These various implementations can include one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) may include machine instructions for a programmable processor, and can be implemented in a high-level procedural and/or object-oriented programming language, and/or in assembly/machine language. As used herein, the term “machine-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

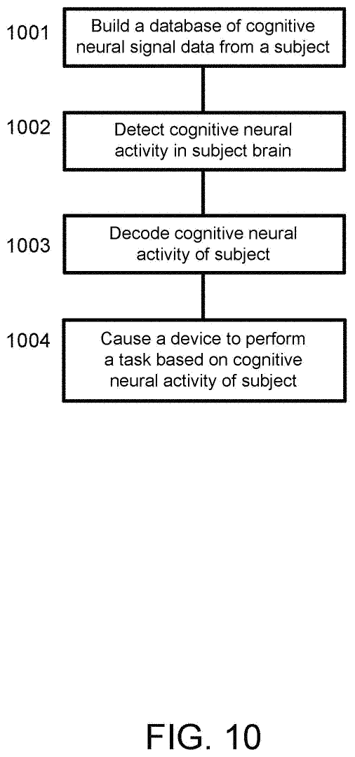

FIG. 10 illustrates one particular logic flow for implementing the system of the present invention. In this embodiment, a database of cognitive neural signal data from a subject may first be built 1001. The database may include neural signal data obtained by way of an implant or any other suitable device that is capable of gathering such information. In one embodiment of the invention, the information itself may relate to the subject's intended movement plan. In other embodiments, the database may include neural signal data from other patients. However, in alternate embodiments the information may relate to a host of other types of data; for instance, intended speech, intended value, or mental/emotional state. Any one form of information may be gathered by an implant or collection of implants; however, in an alternate embodiment, multiple forms of information can be gathered in any useful combination (e.g., intended movement plan and intended value).

In those embodiments of the instant invention in which a database of cognitive neural signal data is compiled from a subject 1001 or subjects 1001, cognitive neural activity can then be detected from the subject's brain 1002. The cognitive neural activity can be detected by the same or different technique and instrumentation that was used to collect information to build the database of neural signal data 1001. Indeed, in one embodiment of the instant invention, a significant period of time may elapse between building the database and using it in connection with the remaining phases in the system logic 1002, 1003, 1004.

In many examples, the data that is collected may be labelled during the training process. For instance, data that is identified as related to a specific effector or action may be incorporated with a label along with other metadata from the subject for example. In various examples, the neural activity would be processed in given time windows as disclosed herein, and filtered out to identify the relevant signals. For instance, if a subject was asked to raise their shoulder, a time window with a few seconds of neural data would be recorded. Potentially, a spike may be identified above a threshold in one of the single units of the array. That spike may then be a set of data that is labeled with the action or instruction. This will allow for training data that can be applied to machine learning algorithms herein.

Once cognitive neural activity from the subject brain is detected 1002, the cognitive neural activity may be decoded 1003. The decoding operation may be performed by any suitable methodology. In one embodiment of the present invention, a Bayesian algorithm on a family of Haar wavelet coefficients (as described in greater detail in the Experimental Results, below) may be used in the decode. A device may then be caused to perform a task based on the cognitive neural activity of the subject 1004.

Although only a few embodiments have been described in detail above, other modifications are possible. For example, the logic flow depicted in FIG. 10 does not require the particular order shown, or sequential order, to achieve desirable results.

### EXAMPLES

The following examples are provided to better illustrate the claimed invention and are not intended to be interpreted as limiting the scope of the invention. To the extent that specific materials or steps are mentioned, it is merely for purposes of illustration and is not intended to limit the invention. One skilled in the art may develop equivalent means or reactants without the exercise of inventive capacity and without departing from the scope of the invention.

### Example 1: Partially Mixed Selectivity in Human Posterior Parietal Association Cortex

The posterior parietal cortex (PPC) has been found to have units selective for a variety of motor variables, but the organization of these representations is still unclear. Here, it was tested how a diverse set of movements are coded within a 4×4 mm patch of the anterior intraparietal area (AIP) of PPC in a tetraplegic human subject. These movements included imagined and attempted movements of the left and right hand and shoulder. Neurons exhibited mixed selectivity to these movements, indicating overlapping multidimensional representations. However, there was also considerable structure within the representations, with body parts functionally segregated, limiting representational interference between body parts. These results show that signals in PPC are highly distributed but still structured, with a small patch of cortex representing many effectors and strategies. This organization is advantageous for prosthetics allowing a single recording array to decode movements of a large extent of the body.

### Experimental Procedures

### Subject

Subject NS has a C3-C4 spinal lesion (motor complete), having lost control and sensation in her hands but retaining movements and sensations in her shoulders.

### Task Procedure

For all tasks the subject sat in a lit room ˜70 cm from a 27-inch LCD screen. No eye fixation was required or enforced.

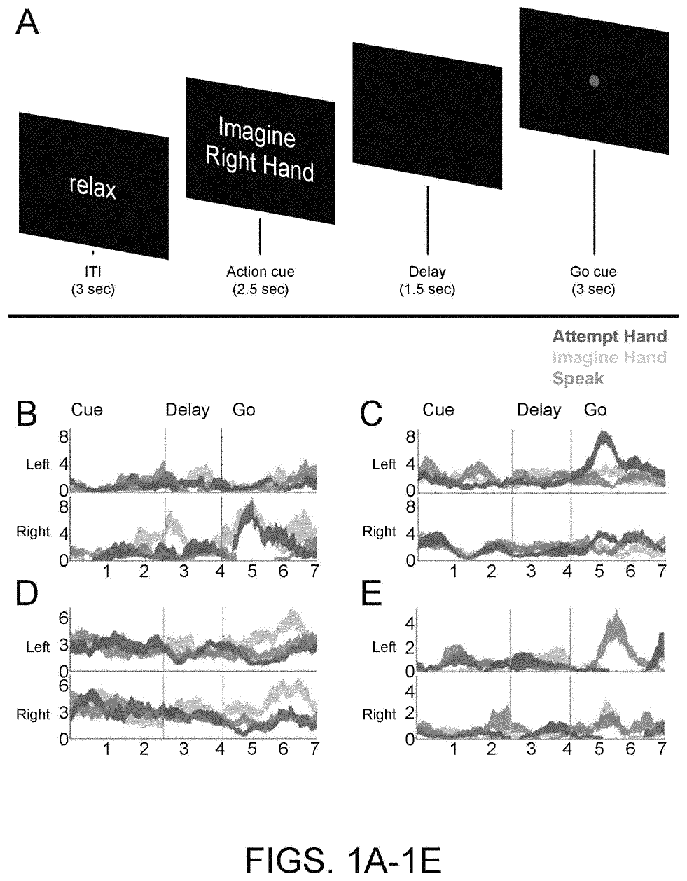

Several versions of a delayed movement task were constructed to determine the extent of tuning to control strategy within the neural populations recorded from AIP. The first task was a text-based task (FIG. 1A). In the first phase the subject was cued for 2.5 seconds what strategy (imagine or attempt), side (left or right), and body part (hand or shoulder) to use, e.g. attempting to squeeze the right hand. In total there were eight possible actions which were randomly interleaved on a trial by trial basis. Hand movements were hand squeezes while shoulder movements were shoulder shrugs. After a delay of 1.5 seconds, the subject was cued to actually perform the cued action for about 3 seconds. Between each trial there was a 3 second inter-trial interval (ITI). This task was used to determine the initial level of tuning for the different strategies when using different effectors. For the version of the task with the eight conditions (imagined/attempted movements of the left/right hand/shoulder), we ran 64 trials (8 trials per condition) on each session. This task was run over the course of 4 non-consecutive days. In total 357 units were recorded across the four recording sessions, assuming independent populations between recording days.

We modified the delay movement task by adding “speak left” and “speak right” as two actions unrelated to any hand or shoulder movements. To avoid exhausting the subject we minimized the number of conditions by splitting sessions into either hand or shoulder movements exclusively. There were 6 total possible commands (Imagine Left, Imagine Right, Attempt Left, Attempt Right, Speak Left, Speak Right) in a session that were randomly interleaved trial by trial. Three sessions were recorded for the hand and the shoulder separately, with each session containing 72 trials (12 trials for each condition). In total 299 units were recorded for sessions using the hand while 228 units were recorded for sessions using the shoulder, once again assuming independent populations between recording days.

Another version was identical in form to the first task looking at imagined and attempted movements of the left and right hand and shoulder, but replacing shoulder shrugs with rigidly raising the arm instead. This allowed us to look exclusivelys at body parts below the level of injury, removing possible confounding effects from one limb being above the level of injury. 6 sessions run over the course of 6 non-consecutive days were recorded, with each session containing 64 trials (8 trials per condition). In total 629 units were recorded across the sessions.

### Recordings

Single unit spikes and firing rates were recorded from a 96 channel Blackrock Neuroport array (electrode length 1.0 mm) implanted in AIP. As a control for executed shoulder movements, an EMG was attached to subject NS's right shoulder to confirm there was no spurious muscle activity when the subject was imagining shoulder movements.

### Analysis

Unit selection: In order to minimize interference from noise, only spikes with a negative deflection exceeding 4.5 standard deviations below baseline were recorded. Units with mean firing rates less than 1.5 Hz were excluded from the analysis as well so that low firing rate effects would be minimized.

Linear analysis: For the first piece of analysis we wanted to determine which movement conditions each unit was tuned to as well as the strength of that tuning. To do this, we fit linear models to each unit's firing rate data. For each unit, the baseline intertrial interval (ITI) activity was subtracted from the “Go” phase firing rate and then fit as a linear function of each of the 8 possible movement conditions independently. We identified units as being tuned to a condition if the slope of their linear fit to the condition (beta value) was statistically significant (p<0.05). This method was used over ANOVA to determine whether a unit was tuned to capture the strength of a unit's tuning to each condition independently as well as the significance of that tuning. ITI activity was taken as the window of activity from 1 second after ITI phase onset to 2.5 seconds after onset (1.5 seconds total) while “Go” activity was taken from the first 2 seconds of the “Go” phase. We selected these time ranges to ensure that the activity used in the analysis was reflective of the expected condition and not the continuation of activity from a previous trial or phase. These time ranges and smoothing windows were used for all other analysis methods as well.

Area under receiver operating curve (AUC) analysis: In addition to the significance of a unit's tuning to a condition, we also wanted to measure the strength of a unit's firing rate signal under a condition relative to its resting/baseline firing rate. To do this, we used the receiver operating characteristic analysis from signal detection theory. For each unit and each movement condition, we computed the classification performance in separating the “Go” phase activity of a neuron from its activity during the ITI phase using just the firing rate with a range of firing rate thresholds, generating a receiver operating curve showing classification performance as a function of threshold. The area under this curve was then computed as a measure of the information content and strength of the tuning of the unit to the movement condition. The AUC values can range from about 0.5 to 1, with 1 indicating perfect tuning (i.e. perfect separation of the firing rates in the “Go” phase from the ITI phase for that condition) and 0.5 indicating no separation (i.e. firing rates of the two phases completely indistinguishable from each other, resulting in purely chance performance for the classifier). The significance and confidence intervals were computed by bootstrapping.

MANOVA of firing rates: To examine the effect of the different motor variables on firing rate patterns across the population we performed a MANOVA test. The baseline firing rate of each neuron (taken during the intertrial interval) was subtracted from the firing rate of the neuron during the movement phase, and this baseline-subtracted firing rate was used in the test. All units were used in the test (regardless of whether they showed tuning to a variable or not).

Degree of specificity: Given the idiosyncratic tuning behavior observed in the single units, we wanted to characterize the tuning of a unit to one condition over its opposite (e.g. left vs right, imagine vs attempt, hand vs shoulder). To do this, we used the degree of specificity as a measure of a unit's specificity to one condition over its opposite. The degree of specificity was defined as a unit's tuning to one condition relative not to its baseline ITI activity but rather to its response to the opposite condition, e.g. a unit's response to the attempt strategy relative to its response to the imagine strategy. Only units significantly different between ITI and Go activity for either or both of the conditions being studied were included, e.g. units tuned to either the strategy or both. We computed this by using the beta values from the linear analysis as measures of the neural representations of each condition. For each unit, we computed the normalized absolute difference in the beta values between opposite conditions. The normalized absolute difference value measures how specific a unit is to one condition over its opposite on a scale of −1 to 1. A value of 1 indicates a significantly higher firing rate for the condition compared to the opposite condition while a value of −1 indicates a significantly higher firing rate for the opposite condition compared to the primary condition in question. A value of 0 indicates no specificity or significant difference in activity between the two conditions.

Correlation between neural representations: we wanted to study the similarity of the neural responses for each condition. To do this, we used the beta values fit from the linear analysis as a measure of the representation of the neural responses at the population level. We correlated the beta values of each of the movement conditions to each other to measure the similarity in neural space of each movement. In this analysis, a high correlation indicates a large degree of overlap between the two movement representations while a low correlation indicates a lower degree of overlap.

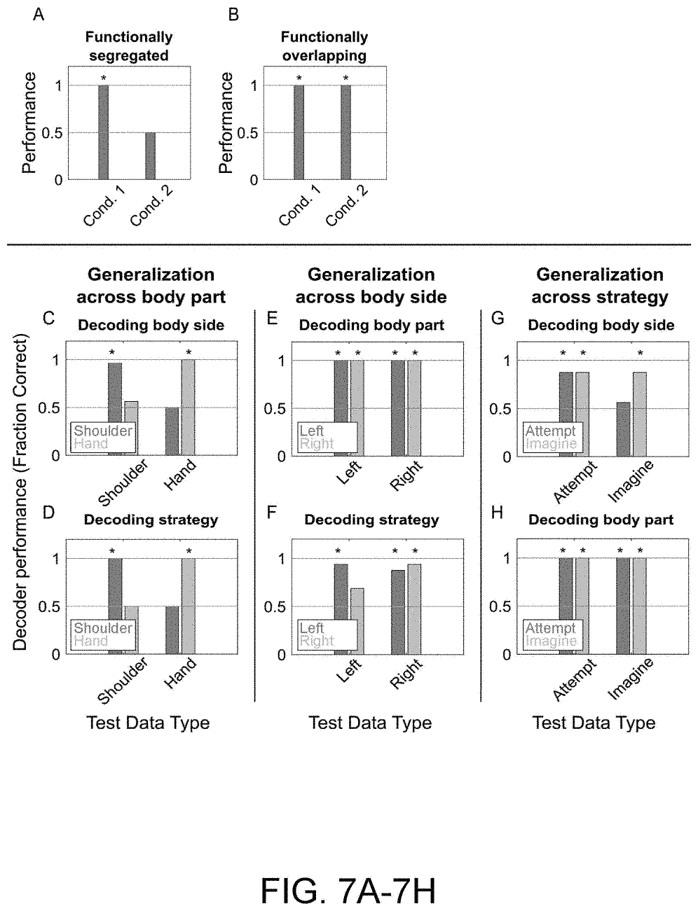

Decoder analysis: To test whether the population contained an abstract representation of each variable, we trained a linear classifier on half of the data, split by one condition, to classify another condition, and tested its performance on the half of the data not used for training. The classifier was also tested in classifying between the third condition to ensure that the classifier was indeed learning how to distinguish between the trained condition. In the case of the analysis for FIG. 7C, for example, the data was split into shoulder movement trials and hand movement trials. For the blue bars, a linear classifier (diagonal linear discriminant type) was trained on the data from trials involving shoulder movements. For features, we used firing rates from the first 2 seconds of the “Go” phase for units with significant tuning to any of the eight movement conditions. The classifier was then tested on shoulder movement data (in-sample, by leave-one-out cross-validation) and hand movement data (out-of-sample). The classifier was also tested on its ability to classify strategy (the third condition) as a control to verify the decoder was indeed learning how to identify the body side (not shown in the Figures), with all such control tests resulting in chance performance.

Confusion matrix: To determine the differentiability of each of the movement conditions from each other, we trained a decoder on the firing rate data and computed how often the decoders incorrectly classified one condition as another. The decoder (diagonal linear discriminant type) was trained on the firing rates during the first 2 seconds of the “Go” phase, learning to identify each of the eight movement conditions from each other. Only units with significant tuning to any of the eight movement conditions were used. Firing rate data was pooled across days, with trials of the same condition shuffled randomly to artificially create additional trials. To generate the confusion matrix, we held out one trial of each condition from the training set (selecting the trial prior to pooling to avoid contamination) and computed the percent of the time each condition was misclassified, keeping track of what it was misclassified as. This process was repeated 100 times (training 100 decoders on different, randomly sampled data sets).

### Results

We compared neural responses of attempted and imagined actions of the hand or shoulder on the right and left side of the body. For hand movements the subject squeezed their hand into a first and for shoulder movements the subject shrugged. Shoulder shrugs are a staple of the subject's behavioral repertoire being a primary method to operate her motorized wheelchair. For imagined movements, the subject was instructed to visualize her limb performing the instructed action, while for attempted movements, the subject was instructed to send the appropriate motor command to move the instructed limb. In the case of shoulder movements, attempted movement resulted in overt motor execution, while for the hand, there was no resulting movement because of paralysis. For the shoulder, we confirmed behavioral compliance by measuring the presence of trapezius EMG activity during attempted but not imagined movement. We used a delayed movement paradigm (FIG. 1A). Following an inter-trial interval (ITI), the subject was instructed to attempt or imagine movement of the left or right hand or shoulder. This instruction was extinguished during a delay period. A generic go cue, visually identical across trial conditions, prompted movement. During the speech condition, the subject simply said “left” or “right” as instructed. Eight repetitions of each trial type were pseudorandomly inter-leaved such that one repetition of each condition was performed before repeating a condition.

FIGS. 1B-E show several well-tuned examples that highlight how neurons commonly coded for a complex assortment of different condition types. For instance, Example B codes for movements of the right hand, whether or not the movement was imagined or attempted. Example C codes exclusively for attempted movements of the left hand. Example D responds similarly for imagined actions of the left or right hand, but not attempted actions. Example E codes for when NS spoke “left.”

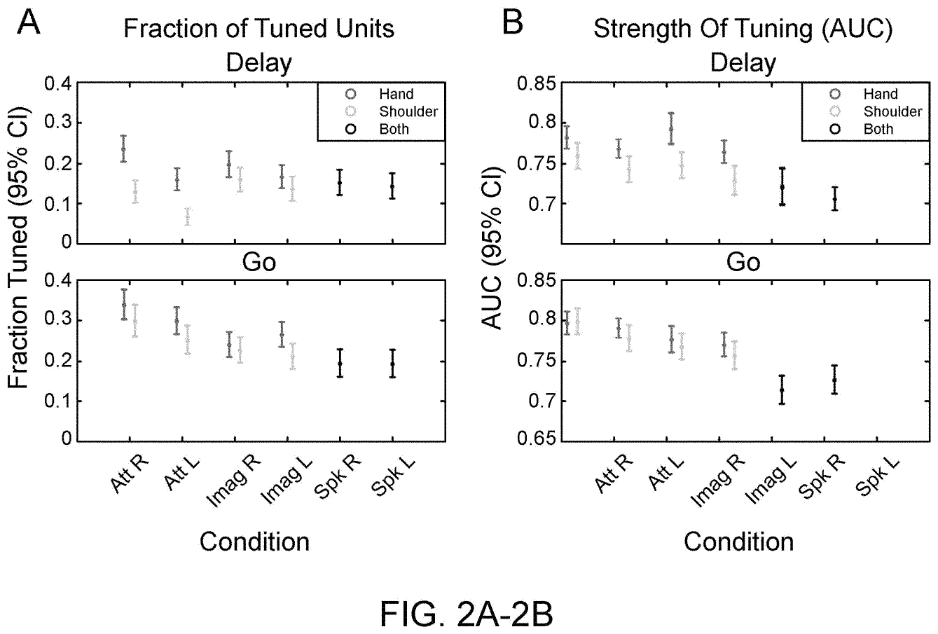

To better understand the strength of tuning in the population to each condition, we fit a linear model to each neuron that explained firing rate relative to baseline (taken as the firing rate during the ITI) as a function of each task condition. A comparison of the significance of the individual beta values showed that a significant proportion of the population was selective for each condition of the hand and shoulder (FIG. 2A) as well as speech. We also examined the magnitude of information content for the tuned units by computing the area under the receiver operating characteristic curve (AUC) generated when comparing the Go/Delay period activity to ITI activity for each condition separately (FIG. 2B). The AUC of the responses to each of the movement conditions were all significant and comparable to each other for hand and shoulder. In contrast, a smaller but still significant percentage of neurons was active for speaking, and the AUC of these responses was smaller when compared to movements of the shoulder and hand on either side of the body (Wilcoxon rank sum test, p<0.05). Assessing the effect of the different motor attributes on firing rate patterns across the population, we found significant differences between hand and shoulder movements (MANOVA p=5.614e-8) and imagined and attempted movements (p=0.0020), and no significant differences between left- and right-sided movements (p=0.2951).

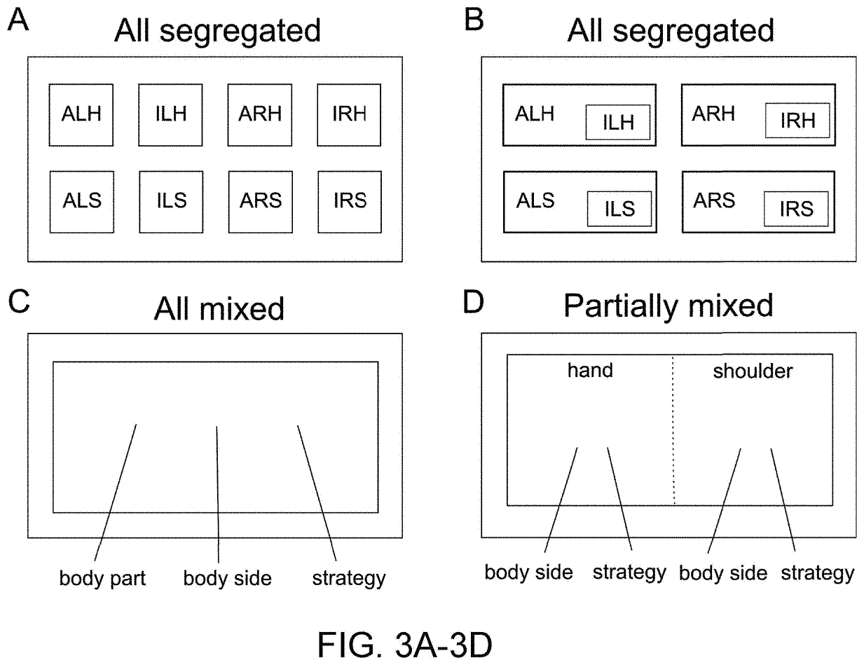

How are these different motor representations coded with respect to each other in the same region of cortex? FIG. 3 shows four possibilities: One, highly specialized sub-populations of neurons could be dedicated to each movement type (FIG. 3A); Two, an organization similar to one, save that some variables are subordinate to others. For instance, imagined movements may be a subset or suppressed version of attempted movements (FIG. 3B); Three, each motor variable class (body part, body side, strategy) could be randomly mixed together (Churchland and Cunningham 2015, Fusi, Miller et al. 2016) (FIG. 3C); Four, some variables may be randomly mixed while others are organized with more structure (partially mixed, FIG. 3D).

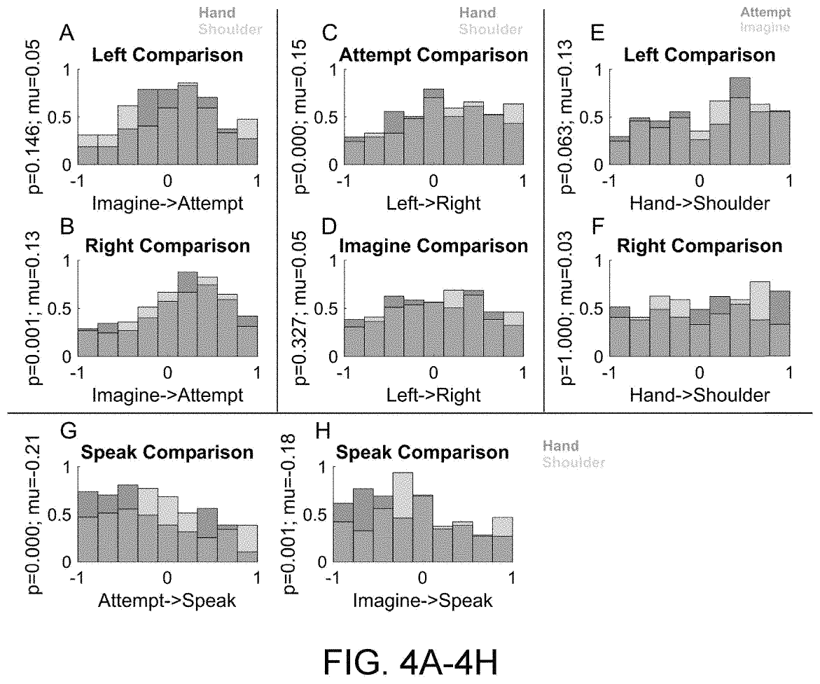

We first performed a degree of specificity analysis (FIG. 4) to determine: one, whether highly specialized sub-populations of neurons are dedicated to each movement type, and, two, whether some variables exist as subsets or suppressed versions of other variables. A specificity index was computed as the normalized difference in beta values between motor variables for each neuron (taken from the linear models described above). Values near zero indicate equivalent neural responses to the two conditions being compared while values near 1 (or −1) indicate exclusive neural responses for one condition. By proposition one, we would expect values to be clustered near 1 (or −1) as e.g. either a neuron is tuned to the right side or the left side. By proposition two, we would expect strong biases such that values would be clustered between 0 and 1 (or 0 and −1) as e.g. a neuron tuned to imagine movement should be better (or equivalently) tuned to attempted movement. Inconsistent with these proposals, we found that specificity values were distributed over the full range (FIG. 4A-F). For instance, despite a small population bias for attempted movements, a sizable proportion of neurons were exclusively, or more strongly activated for imagined movements (FIG. 4AB; see FIG. 1D). The neural representation of motor imagery is not a subset, or less strongly represented version, of motor execution. Likewise, many neurons showed preferential coding for the left hand (FIG. 4CD) even with a population bias for the right hand. There was a strong specificity bias towards imagined or attempted movements of the hand or shoulder over speaking movements (FIG. 4GH). This is expected given that speech tuning is found in a smaller proportion of neurons in a weaker fashion (FIG. 1BC). Of special note, the results are very similar for both movements of the shoulder (above the level of injury) and movements of the hand (below the level of injury) suggesting that the functional organization of motor circuits are preserved even after injury and disuse.

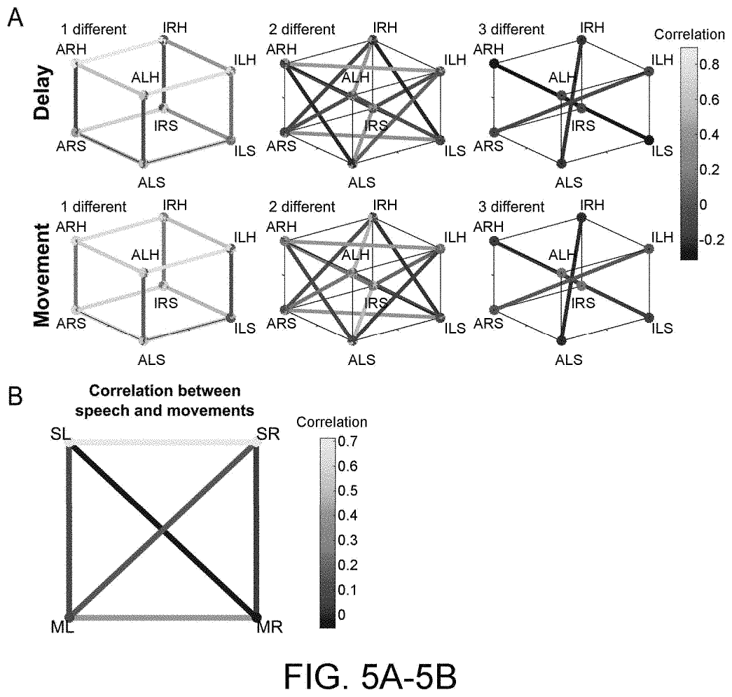

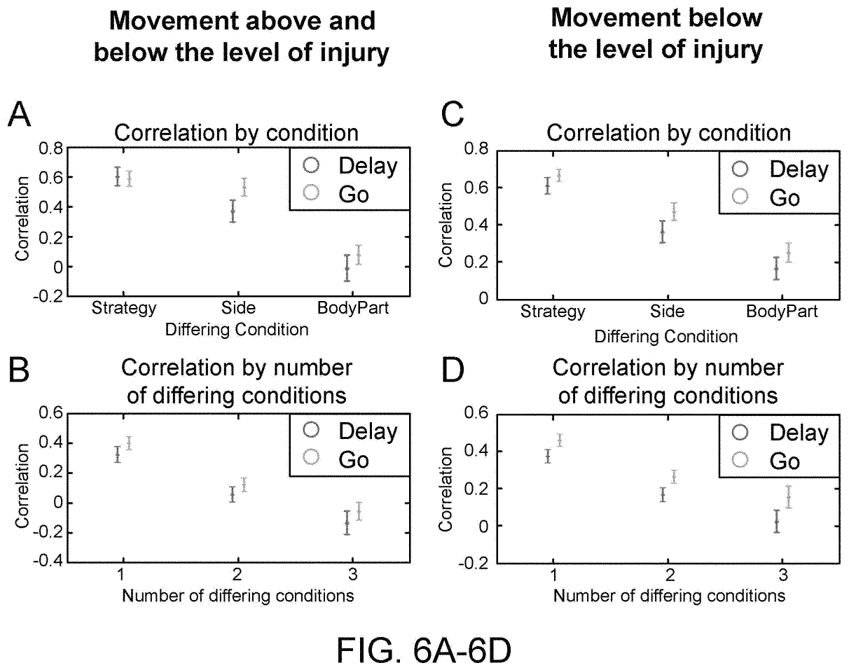

We failed to find complete specialization of function across the population for single units, and the distributed and overlapping nature of responses makes it difficult to find structure in the responses of individual neurons. We therefore turned to population based analyses to more readily identify how the different conditions are encoded with respect to each other. We measured all pairwise correlations between population responses for each condition and looked for systematic structure in how the different motor variables (body part, body side, cognitive strategy) were coded (FIG. 5). Asymmetric relationships between the different variables were immediately apparent. Correlations between conditions that differed in body side or cognitive strategy were high if the comparisons were made within a body part. In stark contrast, correlations between conditions that differed in body part were low even if cognitive strategy and body side were held constant (FIGS. 5A and 6A). Low correlation between body parts was also apparent when comparing speech with shoulder or hand (FIG. 5B). Such low correlations are a signature of network responses that occupy different subspaces thus minimizing crosstalk (Kaufman, Churchland et al. 2014, Churchland and Cunningham 2015). We term this functional segregation of body parts. Further, for a given body part, movements with more shared traits are coded more similarly than movements with fewer shared traits (FIG. 6B). For instance, a neuron tuned to imagined left hand movements was more likely tuned to imagined right hand movements (but not attempted right hand movements). Likewise, a neuron tuned to right hand imagined movements was likely to be tuned to right hand attempted movements (but not left hand attempted movements).

Neural differences between hand and shoulder movements may be driven by the fact that the hand is below the level of injury while the shoulder is above the level of injury: In this case, proprioceptive feedback or long-term effects from the injury might be the primary difference. To address this issue, we replaced shoulder shrugging movements with shoulder abduction (shoulder abduction resulted in no overt motor motion) and repeated the correlation analyses. The results are similar when both body parts are chosen to be below the level of injury (FIG. 6CD). In particular, the largest degree of separation exists between body parts.

Functional segregation of body parts should lead to minimal shared information about other motor variables when compared across body parts. For example, given functional segregation between hand and shoulder, the neural signature that differentiates right from left sided movements for the hand should fail to generalize to the shoulder. We tested for this possibility by looking at patterns of generalization across trained classifiers, e.g. does a classifier trained to differentiate left hand movements from right hand movements generalize to differentiating left shoulder movements from right shoulder movements (and vice versa). Given functional segregation, a classifier trained on condition 1 should fail to generalize to condition 2 (FIG. 7A). Alternatively, for highly overlapping representations, a classifier trained on condition 1 should generalize to condition 2 (FIG. 7B). The results of such an analysis are shown in FIGS. 7C-H. For FIG. 7C, we trained a linear discriminant classifier on all shoulder movement trials to differentiate between left and right-sided movements, regardless of strategy. The decoder performed well within its own training data as expected (leave-one-out cross-validation, FIG. 7C, left blue bar), but performed at chance differentiating left from right-sided movements for hand trials (FIG. 7C, right blue bar). The reverse was true when applying a classifier trained on hand trials to shoulder trials (FIG. 7C, orange bars). Likewise, FIG. 7D shows that a decoder trained to differentiate strategy using shoulder trials failed to generalize to hand trials, and vice versa. In contrast, decoders trained to differentiate strategy or body part were able to generalize and perform well across different body sides (FIGS. 7E-F) and different strategies (FIG. 7GH). Body part differences exhibit functional segregation while cognitive strategy and body side do not.

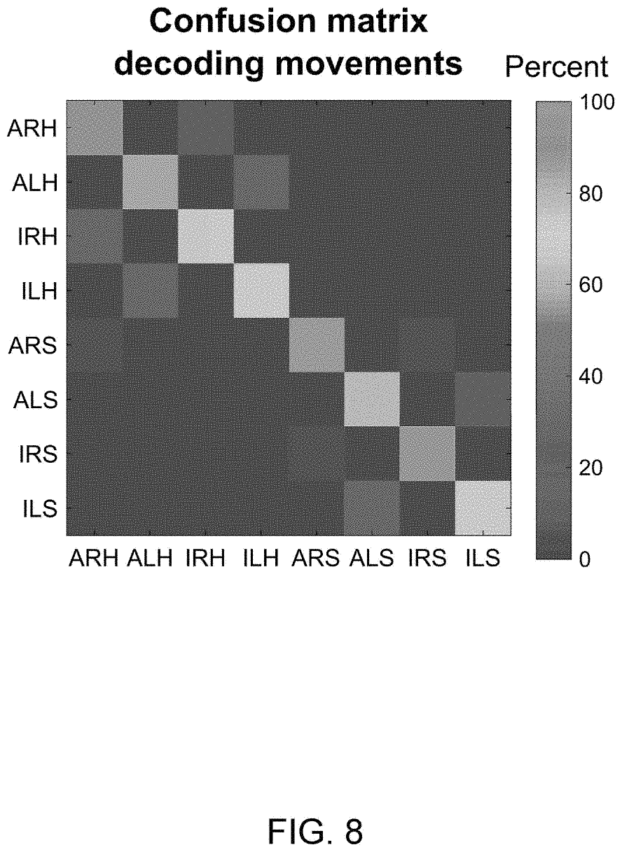

Given that some motor variables are similar in their neural encoding, is it possible to decode the body part, body side, and cognitive motor strategy from the neural population? We constructed a neural classifier to differentiate all conditions (FIG. 8). Cross-validated classification performance was high, however, as expected, misclassification tended to occur between conditions with more variables in common. This is especially true between attempted and imagined movements as predictable from the high degree of similarity in the neural responses (FIG. 6B).

### Discussion

We tested how a variety of motor variables were coded at the level of single neurons in AIP. This allowed us to address several questions about how intent is coded in human AIP and to better understand how the motor variables are coded with respect to each other.

### Effector Specificity in PPC

Classically, the regions around the IPS have been viewed as organizing around the control of different effectors such as the eye, hand, and arm. (Astafiev 2003, Connolly, Andersen et al. 2003, Culham 2003, Prado 2005, Gallivan 2011, Gallivan, McLean et al. 2011). In a recent challenge to the centrality of an effector-based organization, Medendorp and colleagues have found that effector-specificity in the BOLD response of fMRI is much more pronounced between the hand and eye then the hand and other body parts arguing that effectors as such are not differentiated in the planning regions of PPC (Heed, Beurze et al. 2011). In line with these results, we found essentially equivalent numbers of neurons tuned to movements of the hand and shoulder in a small patch of AIP. However, unlike the response at the level of voxels, the neural response to each effector was functionally segregated. Thus, while our results challenge the idea of strict anatomical segregation of effector representations across cortical areas, we do find local functional segregation of effectors within a cortical field. The lack of strict anatomical segregation of effectors may point towards a global topographic organization governed around more behaviorally meaningful aspects of behavior such as manipulation, reaching, climbing, and defense (Graziano and Aflalo 2007, Jastorff, Begliomini et al. 2010) or more basic coordination between effectors. At a minimum, this type of segregation suggests that effector-specificity at the global anatomical scale should be thought of in terms of relative emphasis rather than strict specialization. One exciting aspect of these results is that they open the possibility of decoding movements of many body parts from one small patch of cortex.

### Asymmetric Coding of Motor Variables and Functional Segregation of Body Parts

Recently there has been increased interest in not only the types of variables that are coded in a cortical region, but also how these variables are coded with respect to each other in an effort to understand the underlying logic of the computations performed within a cortical field (Raposo, Kaufman et al. 2014, Fusi, Miller et al. 2016). For instance, several papers have shown that higher cortical areas like PPC and prefrontal cortex may employ a computational strategy by which response variables are randomly mixed (Rigotti, Barak et al. 2013, Raposo, Kaufman et al. 2014). While such a coding scheme can give rise to complex and difficult to interpret representations at the level of single neurons, the population code is information rich and enables simple linear classifiers to decode any variable of interest. In these papers, it was shown that response variables were randomly distributed across neurons, as illustrated in FIG. 3C. Our data provides new insights into understanding population coding by demonstrating that in human AIP certain response features can be seemingly randomly distributed across the population while others are not. In particular, we find that coding for body part is uncorrelated in the sense that across the population, knowing that a neuron is tuned to shoulder movements provides little to no information about whether the neuron is tuned to hand movements (or speech; FIG. 4). This is true even if you know other attributes of the movement, such as whether the movement was imagined or attempted or performed with the right or left side of the body. In contrast, when comparing within the same body part, knowing a neuron is tuned to movements of the right side makes it highly likely that the neuron will be tuned to the left side as well. The same is true for imagined and attempted movements. Thus while some variables seem to be randomly distributed across the population (e.g. body part) the relationship between other variables (e.g. body side, mental strategy) is organized in relationship to a third variable (body part).

The random distribution and uncorrelated relationship of the body part variables is what allows for functional segregation by body part at a population level. The uncorrelated relationship makes it so that information on one body part does not provide information on the other. For example, knowledge of how the body side variables are represented for hand movements is unrelated to how the body side variables are represented for shoulder movements. This effectively segregates hand and shoulder movement representations from each other despite all movements engaging overlapping populations of neurons. Such functional segregation between body parts is very similar in principal to the relationship between planning and execution related activity that has recent been described in frontal motor areas (Churchland, Cunningham et al. 2010, Kaufman, Churchland et al. 2014) where planning activity fails to excite subspaces that are hypothesized to produce muscle output.

Why are some variables functionally overlapping while others are functionally segregated? One possible answer is computational savings. Overlapping activity at the level of the population may be rooted in shared computational resources. For example, many computations related to planning and executing grasps including object affordance processing as well as basic kinematic processing would be similar for the right and left hand. Motor imagery has also been hypothesized to engage internal models used for sensory estimation during overt execution (see below) and thus imagery and execution should rely on largely overlapping computations. Thus despite the potential computational benefits to random mixing of variables (Fusi, Miller et al. 2016), the computational savings of overlapping resources for certain classes of computations may outweigh losses in the total information the population encodes.

Another possibility is that the highly overlapping representations provides part of the neural substrate through which transfer of learning occurs. Motor skills learned with one hand frequently result in improvements in performance with the other hand (Amemiya, Ishizu et al. 2010). Likewise, use of motor imagery is found to improve performance during motor execution (Dickstein 2007). One possibility is that overlapping networks would be able to facilitate this sort of transfer of learning. For example, repeatedly imagining a movement with the right hand would recruit a similar network as executing a movement with the right hand, making any neural adaptation from learning the movement more likely to transfer between the strategies.

A point of note is that the movements selected in this study (hand squeezes and shoulder shrugs) are not necessarily the best exemplars of movements of the respective body parts. Different combinations of hand or shoulder movements may have slightly more or less overlap. Better understanding how different exemplars of movements across different effectors relate will be important in understanding the functional organization of motor actions in AIP.

Attempted and Imagined Movements in Human AIP after Long-Term Injury.

In this study we looked at neural coding of imagined and attempted actions above and below the level of injury in a paralyzed individual. By current theory, imagined movements may represent the simulation of an internal model of the arm, a model that also forms the basis for sensory estimation during overt forms of behavior (Jeannerod 1995, Gail and Andersen 2006, Mulliken, Musallam et al. 2008). In broad strokes, this theory predicts that neural representations of imagined and overt movements should have a high degree of similarity given the shared neural substrate, but also be different given the absence of movement during imagery (Jeannerod 1995, Munzert, Lorey et al. 2009). Our results support this view insofar as we demonstrate the high degree of functional overlap between imagined and attempted movements. However, we also show neural differences between imagined and executed movements persist and are highly similar even after long-term injury and disuse (see FIGS. 4 and 5). Such a preserved distinction does not immediately follow from the proposal that the primary difference between imagined and executed movements is the actual movement itself (Jeannerod 1995). Further, the patterns of similarities and differences in how the population codes mental strategy and body side—for instance, the preference for attempted over imagined movements for the right but not left side of the body (FIG. 4A versus 4B)—demonstrate that higher-order population structure is conserved following injury. This suggests that preservation of motor intention signals enables successful BMI functionality many years post-injury (Aflalo, Kellis et al. 2015). A better understanding of how different cortical subregions maintain representations of motor intent post-injury may help inform choice of implant sites as a function of time post injury.

These results demonstrate for the first time that networks activated during attempted actions are highly overlapping with networks activated during imagined actions at the level of populations of individual neurons, and that the correspondence between actions is body part specific (hand and shoulder). However, there is a symmetry in our results such that networks activated during right hand actions are highly overlapping with networks activated for left hand actions, and the correspondence between right and left actions are strategy specific (e.g. right-side actions look more like left-side actions using the same strategy). In other words, the relationship between imagined and attempted actions is similar in basic form to the relationship between left and right sided actions although the degree of overlap is greater for strategy.

The current experiment was performed in the larger context of a brain-machine interface clinical (BMI) trial. We have previously shown that a paralyzed subject can use motor imagery to control a robotic limb (Aflalo, Kellis et al. 2015). Is the use of motor imagery the best method for the user of a BMI to modulate their own neural activity? Alternatively, it is possible that attempted movements somehow better engage or otherwise enable the subject to control an external device. Here we show that the distinction between imagined and attempted actions is preserved, even in limbs for which no movement is possible. Future work is needed to determine whether these differences translate into performance differences during closed-loop neural control.

### Orofacial Coding in Human AIP.

We included speech conditions in which the subject spoke “left” and “right” as a third fundamentally different movement. A smaller proportion of neurons were tuned more weakly to speech acts, demonstrating that not all actions are coded in an equivalent manner in AIP (FIGS. 2 and 4). This task was not designed to understand the functional significance of “speech” tuned units, but one possibility is that these neurons code for orofacial movements and may form the building blocks for more complex coordinated movements of behavioral relevance such as coordinated movement of the hand to the mouth for feeding or tearing open a bag of chips with your mouth. It is also possible that such orofacial tuning coordinates “grasping” actions performed with the mouth (Jastorff, Begliomini et al. 2010).

While the description above refers to particular embodiments of the present invention, it should be readily apparent to people of ordinary skill in the art that a number of modifications may be made without departing from the spirit thereof. The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

### Selected Embodiments

Although the above description and the attached claims disclose a number of embodiments of the present invention, other alternative aspects of the invention are disclosed in the following further embodiments.

Embodiment. A method, comprising:

detecting, using a detector, neural activity in an area of the brain of a subject and outputting a cognitive signal representative of the neural activity;

processing the cognitive signal to determine a sub-location of the area associated with the cognitive signal;

determining a body part of the subject associated with the sub-location of the area of the brain;

determining a task to perform associated with the cognitive signal for an external device based on at least the body part associated with the sub-location and a level of cognitive signal detected at the first sub-location;

generating a control signal to the external device to perform the task.

The method of an embodiment above, further comprising:

determining whether the sub-location is associated with an attempted movement of the body part;

wherein determining the task to perform is further based on whether the sub-location is associated with an attempted movement.

The method of an embodiment above, further comprising:

determining whether the sub-location is associated with an imagined movement of the body part;

wherein determining the task to perform is further based on whether the sub-location is associated with an imagined movement.

The method of an embodiment above, further comprising:

determining whether the sub-location is associated with a left sided movement of the body part; and

wherein determining the task to perform is further based on whether the sub-location is associated with a left side movement of the body part.

The method of an embodiment above, wherein the sub-location is a single unit neuron.

The method of an embodiment above, wherein the sub-location is latent subspace of the neural population derived as a weighted combination of neural activity.

The method of an embodiment above, wherein the detector is an electrode array, an optogenetic detector and system, or an ultrasound system.

The method of an embodiment above, wherein the step of determining a body part of the subject associated with a first sub-location further comprises, determining a body party previously associated with that sub-location for the subject.

The method of an embodiment above, wherein the step of determining a body part previously associated with that sub-location for the subject comprises instructing the subject to perform a task associated with the body part, and processing the cognitive signals output by the electrode array within a time window of the instructing the subject to perform the task.

The method of an embodiment above, wherein the sub-location is identified by identifying an electrode in the electrode array that processes a cognitive signal above a threshold.

Embodiment. A system comprising:

an electrode array comprising a plurality of electrodes configured to detect neural activity of at least one neuron of the brain of a subject and output a cognitive signal representative of the neural activity;

a memory containing machine readable medium comprising machine executable code having stored thereon instructions;

a control system coupled to the memory comprising one or more processors, the control system configured to execute the machine executable code to cause the one or more processors to:
detecting, using an electrode array, neural activity in an area of the brain of a subject and outputting a cognitive signal representative of the neural activity;
processing the cognitive signal to determine a sub-location of the area associated with the cognitive signal; and
determining a task to perform of the subject based on at least a body part of the subject associated with the sub-location.

The system of an embodiment above, wherein the task is an attempted movement of a right hand or right shoulder.

The system of an embodiment above, wherein the task is an imagined movement of a left hand or left shoulder.

The system of an embodiment above, wherein the external device is a prosthesis.

The system of an embodiment above, wherein the sub-location is in the anterior intraparietal area.

The system of an embodiment above, wherein the sub-location is in the posterior parietal cortex.

The system of an embodiment above, wherein the determining a task to perform based on at least a body part of the subject associated with the sub-location is determined using a linear discriminate classifier.

A system comprising:

an electrode array comprising a plurality of electrodes configured to detect neural activity of at least one neuron of the brain of a subject and output a cognitive signal representative of the neural activity;

a memory containing machine readable medium comprising machine executable code having stored thereon instructions;

a control system coupled to the memory comprising one or more processors, the control system configured to execute the machine executable code to cause the one or more processors to:
detecting, using an electrode array, neural activity in an area of the brain of a subject and outputting a cognitive signal representative of the neural activity;
processing the cognitive signal to determine a task for an external prosthesis to perform based at least on a body part associated a set of sub-locations of the area that detect a threshold level of cognitive signal within a time period;
sending a control signal to move the external prosthesis if the set of sub-locations is associated with attempted movement more than imagined movement.

A system comprising:

an electrode array comprising a plurality of electrodes configured to detect neural activity of at least one neuron of the brain of a subject and output a cognitive signal representative of the neural activity;

a memory containing machine readable medium comprising machine executable code having stored thereon instructions;

a control system coupled to the memory comprising one or more processors, the control system configured to execute the machine executable code to cause the one or more processors to:
detecting, using an electrode array, neural activity in an area of the brain of a subject and outputting a cognitive signal representative of the neural activity;
processing the cognitive signal to a task for an external device to perform based at least on a body party associated a set of sub-locations of the area that detect a threshold level of cognitive signal within a time period and whether that body part is associated with attempted movement in that set of sub-locations;

The method of an embodiment above, wherein the cognitive signal describes an intended goal of the subject.

The method of an embodiment above, wherein the cognitive signal describes a reach goal, an expected value, speech, abstract thought, executive control, attention, decision, and motivation.

A method, comprising:

detecting, using a detector, neural activity in an area of the brain of a subject and outputting a cognitive signal representative of the neural activity;

processing the cognitive signal to determine a sub-location of the area associated with the cognitive signal;

determining a superordinate variable associated with the sub-location of the area of the brain;

determining a task to perform associated with the cognitive signal for an external device based on at least the superordinate variable associated with the sub-location and a level of cognitive signal detected at the first sub-location; and

generating a control signal to the external device to perform the task.

The method of an embodiment above, wherein the superordinate variable is a body part.

The method of an embodiment above, further comprising processing the cognitive signal to determine a subordinate variable associated with the cognitive signal at the sub-location based on the determined superordinate variable associated with the sub-location.

The method of an embodiment above, wherein determining the subordinate variable, further comprises determining a spatial distribution and intensity of neural activity within the sub-location.

The method of an embodiment above, wherein the subordinate is one of cognitive strategy or body side.

A system comprising:

an electrode array comprising a plurality of electrodes configured to detect neural activity of at least one neuron of the brain of a subject and output a cognitive signal representative of the neural activity;

a memory containing machine readable medium comprising machine executable code having stored thereon instructions;

a control system coupled to the memory comprising one or more processors, the control system configured to execute the machine executable code to cause the one or more processors to:
detecting, using an electrode array, neural activity in an area of the brain of a subject and outputting a cognitive signal representative of the neural activity;
designing an algorithm that processes the cognitive signal to a task for an external device to perform leveraging the partially mixed selectivity structure described above as to improve performance/efficiency;

The method of an embodiment above, wherein the algorithm describes one where decoder variables learned for certain variables A and B are regularized to comport with the known mixing structure between A and B.

The method of an embodiment above, wherein the algorithm describes one where initial seeds for the decoder parameters of variable A are selected based on the parameters for a decoder B based on the known mixing structure between A and B.

The method of an embodiment above, wherein the algorithm describes one where decoding parameters for a subset of decodable variables X are updated based on observed changes in the relationship between neural activity and decodable variables Y in order to preserve the known mixing structure between variables. For example, parameters changes made to variable A in response to the loss of a neural channel could be propagated to parameters B, C, D, etc. based on the known mixing structure between the variables.

The method of an embodiment above, wherein the algorithm describes one where the training or prediction stages utilize the known internal structure of the variables. For example, Bayesian hierarchical modeling or deep networks. Decoding along one variable first (e.g. body part) and then using the result of that decoder to inform the decoding process for subsequent variables (e.g. strategy, body side).

A system comprising:

an electrode array comprising a plurality of electrodes configured to detect neural activity of at least one neuron of the brain of a subject and output a cognitive signal representative of the neural activity;

an electrode array comprising a plurality of electrodes configured to stimulate neural activity of at least one neuron of the brain of a subject and input a cognitive signal representative of the neural activity;

a memory containing machine readable medium comprising machine executable code having stored thereon instructions;

a control system coupled to the memory comprising one or more processors, the control system configured to execute the machine executable code to cause the one or more processors to:
detecting, using an electrode array, neural activity in an area of the brain of a subject and outputting a cognitive signal representative of the neural activity;
stimulating, using an electrode array, neural activity in an area of the brain of a subject and inputting a signal;

The method of an embodiment above, wherein the input signal evokes a specific sensory percept in a way that leverages the partially mixed structure of the recorded population. For example, stimulating to cause a hand sensation without causing a shoulder sensation.

The method of an embodiment above, wherein the input signal sends feedback from training/learning to a specific subpopulation of neurons. For example, sending direct neural feedback about one effector without significantly affecting another effector's representation.

The method of an embodiment above wherein the input signal causes a specific motor movement to occur in a way that leverages the partially mixed structure of the recorded population. For example, stimulating to cause a hand movement without causing a foot movement.

### REFERENCES

Aflalo, T., S. Kellis, C. Klaes, B. Lee, Y. Shi, K. Pejsa, K. Shanfield, S. Hayes-Jackson, M. Aisen, C. Heck, C. Liu and R. A. Andersen (2015). “Decoding motor imagery from the posterior parietal cortex of a tetraplegic human.” Science 348(6237): 906-910.
Amemiya, K., T. Ishizu, T. Ayabe and S. Kojima (2010). “Effects of motor imagery on intermanual transfer: A near-infrared spectroscopy and behavioral study.” Brain Research 1343: 93-103.
Andersen, R. A. and C. A. Buneo (2002). “Intentional maps in posterior parietal cortex.” Annual Review of Neuroscience 25: 189-220.
Andersen, R. A. and H. Cui (2009). “Intention, Action Planning, and Decision Making in Parietal-Frontal Circuits.” Neuron 63(5): 568-583.
Andersen, R. A., G. K. Essick and R. M. Siegel (1987). “Neurons of area 7 activated by both visual stimuli and oculomotor behavior.” Experimental Brain Research 67: 316-322.
Astafiev, S. V., Shulman, G. L., Stanley, C. M., Snyder, A. Z., Van Essen, D. C., and Corbetta, M. (2003). “Functional organization of human intrapaietal and frontal cortex for attending, looking, and pointing.” Journal of Neuroscience 23: 4689-4699.
Balint, R. (1909). “Seelenlahmung des “Schauens,” optische Ataxie, raumliche Storung der Aufmerksamkeit.” Monatsschr. Psychiatr. Neurol. 25: 51-81.
Beurze, S. M., de Lange, F. P., Toni, I., and Medendorp, W. P. (2009). “Spatial and effector processing in the human parietofrontal network for reachces and saccades.” J. Neurophsiol. 101: 3053-3062.
Churchland, M. M. and J. P. Cunningham (2015). “A Dynamical Basis Set for Generating Reaches.” Cold Spring Harbor Symposia on Quantitative Biology: 024703.
Churchland, M. M., J. P. Cunningham, M. T. Kaufman, S. I. Ryu and K. V. Shenoy (2010). “Cortical preparatory activity: representation of movement or first cog in a dynamical machine?” Neuron 68(3): 387-400.
Connolly, J. D., R. A. Andersen and M. A. Goodale (2003). “FMRI evidence for a ‘parietal reach region’ in the human brain.” Exp Brain Res 153(2): 140-145.
Culham, J. C., Danckert, S. L., DeSouza, J. F. X., Gati, J. S., Menon, R. S., and Goodale, M. A. (2003). “Visaully guided grasping produces fMRI activation in dorsal but not ventral stream brain areas.” Exp. Brain Res. 153: 180-189.
Dickstein, R. (2007). “Motor Imagery in Physical Therapy Practice.” Journal of the American Physical Therapy Association 87(7): 942-953.
Fusi, S., E. K. Miller and M. Rigotti (2016). “Why neurons mix: high dimensionality for higher cognition.” Current Opinion In Neurobiology 37: 66-74.
Gail, A. and R. A. Andersen (2006). “Neural dynamics in monkey parietal reach region reflect context-specific sensorimotor transformations.” J Neurosci 26(37): 9376-9384.
Gallivan, J. P., D. A. McLean, F. W. Smith and J. C. Culham (2011). “Decoding effector-dependent and effector-independent movement intentions from human parieto-frontal brain activity.” The Journal of Neuroscience 31(47): 17149-17168.
Gallivan, J. P., McLean, D. A., Flanagan, J. R., and Culham, J. C. (2013). “Where one hand meets the other: limb-specific and action-dependent movement plans decoded from preparatory signals in single human frontoparietal brain areas.” Journal of Neuroscience 33: 1991-2008.
Gallivan, J. P., McLean, D. A., Smith, F. W. and Culham, J. C. (2011). “Decoding effector-dependent and effector-independent movement intentions from human parieto-frontal brain activity.” Journal of Neuroscience 31: 17149-17168.
Gerardin, E., A. Sirigu, S. Lehéricy, J. B. Poline, B. Gaymard, C. Marsault, Y. Agid and D. Le Bihan (2000). “Partially overlapping neural networks for real and imagined hand movements.” Cerebral Cortex 10(11): 1093-1104.
Graziano, M. S. A. and T. N. Aflalo (2007). “Rethinking cortical organization: moving away from discrete areas arranged in hierarchies.” The Neuroscientist: a review journal bringing neurobiology, neurology and psychiatry 13(2): 138-147.
Heed, T., S. M. Beurze, I. Toni, B. Roder and W. P. Medendorp (2011). “Functional Rather than Effector-Specific Organization of Human Posterior Parietal Cortex.” The Journal of Neuroscience 31(8): 3066-3076.
Heed, T., S. M. Beurze, I. Toni, B. Roder and W. P. Medendorp (2011). “Functional rather than effector-specific organization of human posterior parietal cortex.” Journal of Neuroscience 31(8): 3066-3076.
Hinkley, L. B. N., Krubitzer, L. A., Padberg, J., and Disbrow, E. A. (2009). “Visual-manual exploration and posterior parietal cortex in humans.” J. Neurophsiol. 102: 3433-3446.
Holmes, G. (1918). “Disturbances of visual orientation.” Br J Opthalmol 2: 449-468.
Jastorff, J., C. Begliomini, M. Fabbri-Destro, G. Rizzolatti and G. A. Orban (2010). “Coding Observed Motor Acts: Different Organizational Principles in the Parietal and Premotor Cortex of Humans.” J. Neurophysiol 104: 128-140.
Jeannerod, M. (1995). “Mental imagery in the motor context.” Neuropsychologia 33(11): 1419-1432.
Kaufman, M. T., M. M. Churchland, S. I. Ryu and K. V. Shenoy (2014). “Cortical activity in the null space: permitting preparation without movement.” Nat Neurosci 17(3): 440-448.
Klaes, C., Kellis, S., Aflalo, T., Lee, B., Pejsa, K. Shanfield, K., Hayes-Jackson, S., Aisen, M., Heck, C., Liu, C. and Andersen, R. A. (2015). “Hand shape representations in the human posterior parietal cortex.” J. Neurosci. 35: 15466-15476.
Levy, I., Schluppeck, D., Heeger, D. J., and Glimcher, P. W. (2007). “Specificity of human cortical areas for reaches and saccades.” Journal of Neuroscience 27: 4687-4696.
Mountcastle, V. B., Lynch, J. C., Georgopoulos, A., Sakata, H., Acuna, C. (1975). “Posterior parietal association cortex of the monkey: command functions for operations within extrapersonal space.” J Neurophysiol 38(4): 871-908.
Mulliken, G. H., S. Musallam and R. A. Andersen (2008). “Forward estimation of movement state in posterior parietal cortex.” Proceedings of the National Academy of Sciences 105(24): 8170-8177.
Munzert, J., B. Lorey and K. Zentgraf (2009). “Cognitive motor processes: the role of motor imagery in the study of motor representations.” Brain Res Rev 60(2): 306-326.
Murata, A., Gallese, V., Luppino, G., Kaseda, M., and Sakata, H. (2000). “Selectivity for the shape, size, and orientation of objects for grasping in neurons of monkey parietal area AIP.” J. Neurophysiol. 83: 2580-2601.
Prado, J., Clavagnier, S., Otzenberger, H., Scheiber, C., Kennedy, H. and Pererin, M. T. (2005). “Two cortical systems for reaching in central and peripheral vision.” Neuron 48: 849-858.
Raposo, D., M. T. Kaufman and A. K. Churchland (2014). “A category-free neural population supports evolving demands during decision-making.” Nature Neuroscience 17(12): 1784-1792.
Rigotti, M., O. Barak, M. R. Warden, X. J. Wang, N. D. Daw, E. K. Miller and S. Fusi (2013). “The importance of mixed selectivity in complex cognitive tasks.” Nature 497: 585-590.
Snyder, L. H., A. P. Batista and R. A. Andersen (1997). “Coding of intention in the posterior parietal cortex.” Nature 386(6621): 167-170.
Ungerleider, L. G. and M. Mishkin (1982). Two cortical visual systems. Analysis of Visual Behavior. D. J. Ingle, M. A. Goodale and R. J. W. Mansfield. Cambridge, Mass., MIT Press: 549-585.

### Computer & Hardware Implementation of Disclosure

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

1. A system comprising:
an electrode array comprising a plurality of electrodes configured to detect neural activity of at least one neuron of the brain of a subject and output a cognitive signal representative of the neural activity;
a memory containing machine readable medium comprising machine executable code having stored thereon instructions;
a control system coupled to the memory comprising one or more processors, the control system configured to execute the machine executable code to cause the one or more processors to:
detecting, using an electrode array, neural activity in an area of the brain of a subject and outputting a cognitive signal representative of the neural activity;
processing the cognitive signal with a decoder to determine a task for an external prosthesis to perform by first determining a body part associated with the cognitive signal and after determining the body part associated with the cognitive signal, then determining the body side of the body part associated with the cognitive signal, based on the spatial location and intensity of the neural activity within the area; and
sending a control signal to move the external prosthesis based on the determined task.

2. The system of claim 1, wherein the task is an attempted movement of a right hand or right shoulder.

3. The system of claim 1, wherein the decoder comprises a predictive model.

4. The system of claim 3, wherein the predictive model is a linear discriminate classifier.

5. The system of claim 1, wherein the area comprises a set of single unit neurons.

6. The system of claim 1, wherein the area comprises the anterior intraparietal area (AIP).

7. The system of claim 1, wherein the area comprises the posterior parietal cortex (PPC).

8. The system of claim 1, wherein the control system is further configured to execute the machine executable code to cause the one or more processors to determine a cognitive strategy associated with the cognitive signal after determining the body part associated with the cognitive signal.
